(19) United States
(12) Patent Application Publication
(10) Pub. No.: US 2007/0249038 A1
(43) Pub. Date: Oct. 25, 2007

(54) Microfluidic device for single cell targeted operations

(76) Inventors: Andrea Adamo, Cambridge, MA (US); Klavs Flemming Jensen, Lexington, MA (US); Luigi Adamo, Cambridge, MA (US)

Correspondence Address: Andrea Adamo, 305 Holden Green - apt B, Cambridge, MA, 02138, US

(21) Appl. No.: 11/788,646

(22) Filed: Apr. 20, 2007

Related U.S. Application Data
(60) Provisional application No. 60/794,237, filed on Apr. 21, 2006.

(51) Int. Cl. C12M 3/00

(52) U.S. Cl. 435/287.1

(57) ABSTRACT

Here we describe an integrated microfluidic chip for delivery of fluids inside or in proximity of individual cells or for measurement of physical quantities. In this device, a cell suspension is pumped through a microchannel in which cells are lined up, pushed on a static microneedle or micropipette, and then collected in a reservoir. The channels are microfabricated. Valves can be embedded in the system to allow fluid flow control thus enhancing the efficiency of the system and reducing malfunctioning due to clogging or any other unexpected issues.

### CROSS REFERENCE TO RELATED APPLICATIONS

This application claims benefit of U.S. provisional application No. 60/794,237 filed on Apr. 21, 2006, which is hereby incorporated by reference.

### BACKGROUND OF THE INVENTION

High throughput single cell targeting is currently the limiting step in many fundamental processes in the biomedical sciences, among these are patch clamp and microinjection.

Microinjection is currently the only existing technique that allows efficient introduction into single cells of any kind of material (e.g. lipids, proteins, carbohydrates, nucleic acids, chemicals in general) or structure (e.g. sub-cellular organelles or microfabricated/nanofabricated structures). However, microinjection is at present expensive and extremely slow (it typically takes several minutes for an experienced operator to perform an injection into one cell).

Patch clamp is the most accurate tool to obtain measurements of the electrical activity of single cells. This technique can be performed with manual cell handling or machine assisted cell handling. In both cases the most delicate part of the procedure is that of accurately positioning single cells in proximity to the micropipette used to measure electrical activity at the cell membrane.

Both Microinjection and Patch clamp, as well as a variety of other biomedical applications, would benefit from the ability to rapidly position single cells in a desired location with micrometric precision. The technology disclosed in U.S. Pat. No. 6,846,668 has set some preliminary steps towards an efficient positioning of single cells for the purpose of high throughput microinjection; however, further improvement is needed.

### BRIEF SUMMARY OF THE INVENTION

Here we describe an integrated microfluidic chip for positioning of single cells with micrometric or submicrometric precision and subsequent delivery of fluids inside/in proximity of individual cells or measurement of physical/chemical properties in the cell or in its immediate proximity. In this device, a cell suspension is pumped through a microchannel in which cells are lined up, pushed on a “working area” where a static microneedle or micropipette or sensing probe can be present, and then collected in a reservoir. The channels are microfabricated. Valves can be embedded in the system to allow fluid flow control thus enhancing the efficiency of the system and reducing malfunctioning due to clogging or any other unexpected issues. The microneedle or micropipette or sensing probe used can be externally fabricated and then inserted into the device. The system can be built with low cost materials so that chips can be disposable. The invention is described in detail for the case in which a microneedle is positioned into the “working area” in order to achieve single cell microinjection. Schematics of the system, pictures of the prototype and pictures from successful experiments are provided.

### BRIEF DESCRIPTION OF THE DRAWINGS

FIG. 1 is a schematic of the principle of operation of the present invention (case of the microneedle for microinjection)

FIG. 2 shows a glass microneedle an the injection area in a prototype device built in Polydimethylsiloxane (PDMS) to test the concept for microinjection purposes

FIG. 3 shows a view of a prototype device

FIG. 4 shows an overall view of the packaged prototype device

FIG. 5 shows a cell while performing microinjection with a prototype device. The cell can be recognized because it is stained with a fluorescent vital dye.

FIG. 6 shows a just injected cell in a prototype device. The cell can be recognized because a fluorescent dye has been injected (the cell fluoresces as the needle).

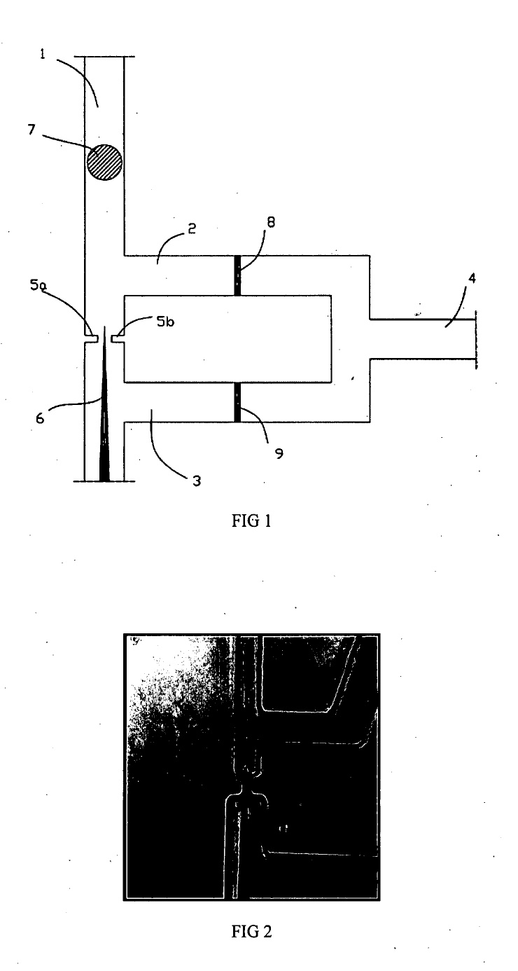

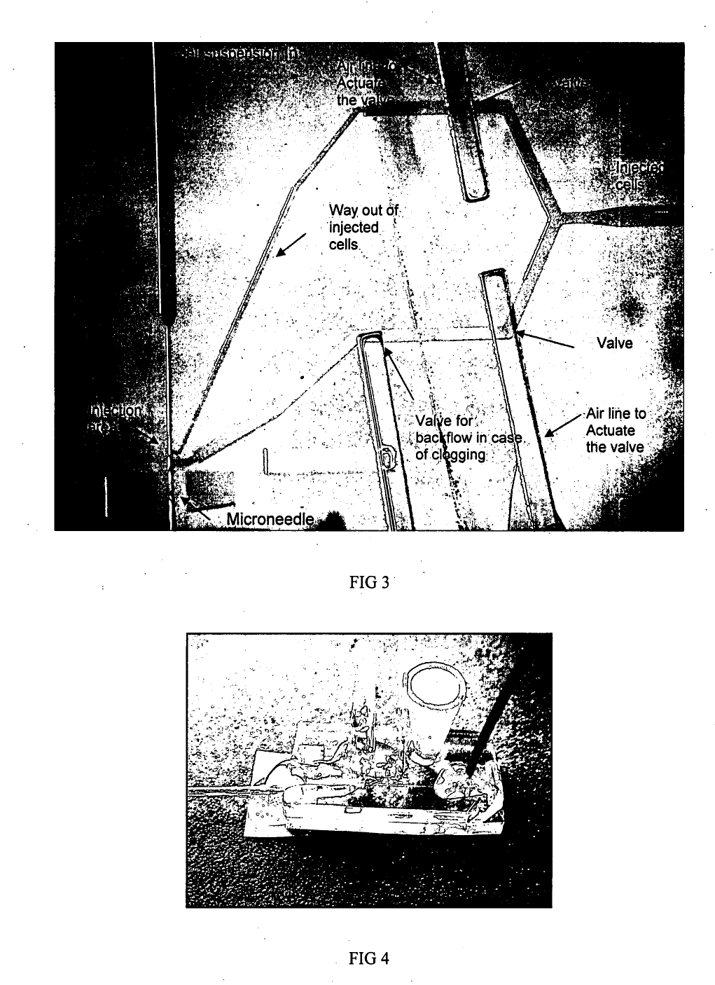

### DETAILED DESCRIPTION OF THE INVENTION

We describe an integrated microfluidic chip for high throughput single cell positioning with micrometric or submicrometric precision with the ability to delivery fluids inside or in proximity to individual cells and record measurements in proximity or inside cells. In this device, a cell suspension is pumped through a microchannel in which cells are lined up, pushed to a “working area” where a static microneedle or micropipette or sensing probe is placed, and then collected in a reservoir. The channels are microfabricated. Valves can be embedded in the system to allow fluid flow control thus enhancing the efficiency of the system and reducing malfunctioning due to clogging or any other unexpected issues. The micro needle or micropipette or sensing system can be externally fabricated and then inserted into the device. The system can be built with low cost materials so that chips can be disposable.

The device concept is most easily described by following the cells in their path through the core of the device in the case in which a needle for microinjection is placed in the “working area”. For this description we'll refer to FIG. 1. Cells are first suspended in an appropriate solution. The solution with suspended cells flows into channel 1 in which the cells are lined up by the channel width and height being comparable to the size of the cells. A microneedle 6 (or micropipette) is placed at the end of channel 1. At this time valve 9 is open and valve 8 closed, therefore the suspension solution flows out via channel 3. When a cell 7 reaches the end of channel 1, it is retained by 5a and 5b. Because of its momentum the cell upon contact with the needle 6 is pierced. At this point the injection is performed. To remove the cell and deliver it to a collection reservoir or to the next processing if the injection is operated on a more complex integrated device, valve 9 is closed and valve 8 opened. In such a way the cell can be taken away through channel 2 and then channel 4. As soon as the cell gets into channel 2, the valves may be switched again (8 closed and 9 opened) restoring the initial conditions, therefore the system is ready to inject another cell. A cell detection system (not shown in the figures) may be present in order to synchronize the microinjection and the valve actuation, or any other required operation, with the presence of the cell. Such a detection system can be of different types. For instance it can be optical, electrical or based on image analysis.

An aspect of novelty of the invention lays in the way cells are moved toward the “working area” and away from it. The presence of channel 3 basically allows the suspension fluid to be drained away while channel 2 allows the delivery of the injected cell to a collection reservoir. Channels 2 and 3 need not merge but the merging improves the system because pressure drops along channel 2 and 3 can be balanced in such a way that upon actuation of the valves, the flow rate remains fairly constant.

The chip (assembly of channels with at least one inlet and one outlet) can be constructed using microfabrication techniques comprising, among the others, polymer microfabrication techniques such as soft lithography. For mass production, techniques as hot embossing or injection molding can be selected. Polymers can be preferred for their low cost and simplicity of manufacturing process. Moreover they can be of advantage if the device, or part of it, is meant to be disposable. The prototype reported in the figures has been constructed with an elastomer (PolyDimethylSiloxane—PDMS). If needed, surface modification of the channels walls can be done to improve wettability or to avoid sticking of the cell membrane to the channel walls.

The channels have size in the order of microns, tens of microns or hundreds of microns. Where the injection, sampling or measurement in general takes place, the dimension of the channels is comparable to the dimension of the targeted cell type, so that the required operation can be executed with precision (cross section with characteristic size in the order of 1 micron to 100 micron or larger depending on the cell type).

The valves are constructed with techniques available in the technical literature and compatible with the chosen microfabrication process. An example is that of valves made with a thin layer of elastomer that can be deformed by applying pressure; the deformation of the elastomer reduces the cross section of the channel to be controlled thus achieving control of the fluidic stream. Beside pneumatic valves, other types of valves can be used depending on the specific material chosen for the chip and the specific microfabrication technique. Valves can be of the normally closed or normally open type, upon the selection, actuation will vary accordingly so that cell handling can be achieved.

If instead of microinjection other operations are to be performed, as patch clamp or other measurement of cell electric potential, an appropriate microneedle or micropipette or sensing probe will be chosen and positioned inside the chip according to the specific needs. According to the specific application or cell type the protrusion of the needle or micropipette or sensing probe into channel 1 will be selected. The protrusion of the needle or micropipette determines the penetration depth of the needle into the cell or the location of the contact between the cell and the micropipette. If needed, the needle or micropipette can be used for withdrawal of fluid.

Microneedles and micropipettes and sensing probes need not be fabricated exclusively according to the art of microfabrication and can be fabricated in other ways and then inserted into the polymeric chip in the location where the injection needs to take place. Here we refer to microfabrication as the art of miniaturization that historically originated from semiconductor industry. The micro-needle or micropipette could be a multi-barrel pulled glass capillary thus enabling simultaneous microinjections of different compounds into the same cell or allowing a combination of different operations on the same cell. Micropipettes and microneedles can be fabricated for instance pulling glass capillaries or with other appropriate procedures. Other materials different than glass can be selected depending on the specific application. Materials can be electrically conductive or not or can have electrically conductive parts. The size of the tip of the microneedle or micropipette can be in the range between 0.05 micron to 100 micron depending on the specific application and the specific cell type.

One of the possible ways to insert and position the microneedle or micropipette into the chip is making use of a micrometer stage and optical microscopy. In the prototype of FIG. 4 the needle placement has involved a first step where the needle is introduced in the appropriate channel and a second step where the needle is appropriately positioned inside the injection area. Because of the different refractive index of air and PolyDimethylSiloxane (PDMS), the first step of the alignment requires tilting of the board with the chip and the micro stages so that the hole for the needle insertion can be clearly seen with the stereo microscope and the needle aligned. After introduction of the needle into the appropriate channel, the board with chip and micropositioners is tilted back to the horizontal position and the needle can be correctly placed in the injection area. At this stage illumination from below enhances the success rate of needle placement. After correct placement the needle is secured to the chip with glue. During the placement care needs to be exercised in the positioning so that protrusion length of the needle or micropipette is controlled.

Experiments were performed with a prototype device. In order to the test the efficiency of the described design we have first tested the ability to generate reversible fluidic streams as predicted by mean of fluorescent micro beads. After optimizing the hydraulic features of the cell handling chip we have tested the compatibility of mammalian cells with the proposed handling system. Hela DME cells cultured in Dulbecco Modified Eagle Medium (DMEM) after detachment with trypsin and resuspension in Phosphate Buffered Solution without Calcium or Magnesium (PBS) were flowed into the chip. Viability analysis of cells flowed through the system by Trypan Blue staining showed 98% viabiliy at one hour as compared to control.

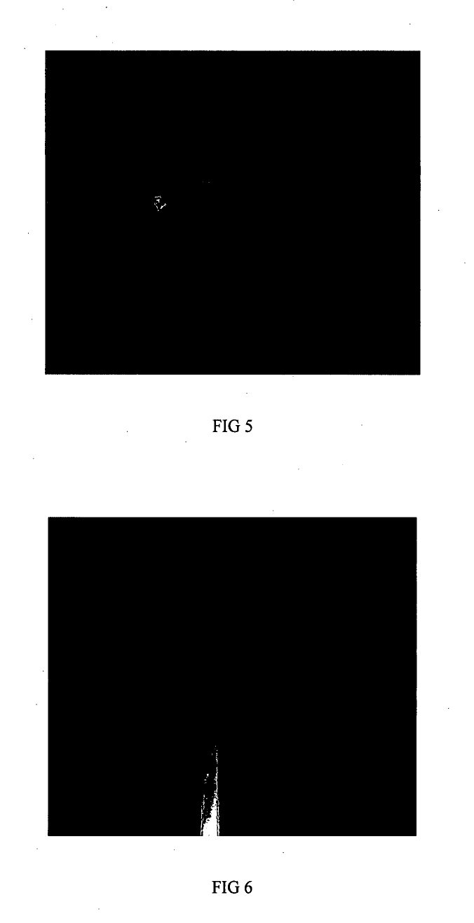

After determining that just the flow through the chip does not significantly alter viability, and that cell positioning with micrometric or submicrometric precision can be achieved we tested the efficiency of our system for the case of microinjection. To this purpose, a microneedle was place in the “working area”, we selected a fluorescent compound unable to cross cell membranes unless microinjected (dextran, tetramethylrhodamine MW 10000, Molecular probes, USA). Hela cells were pierced as expected and successfully injected with the dextran (FIG. 6). Viability of injected cells was assessed first by the absence of cells coloration in the presence of Trypan Blue (it stains dead cells) and after by culturing of injected cells.

The described system can be used to inject into cells or deliver in proximity of a cell any kind of material. The cells are preferably but not exclusively mammalian cells. Materials to be delivered can be, but are not limited to, solutions of lipids, proteins, carbohydrates, nucleic acids and chemicals in general or structures (e.g. sub-cellular organelles or microfabricated/nanofabricated structures) or a mix of any of the above. Other applications relate to measurement of physical quantities of relevance for cells studies (i.e. patch clamp). A non exhaustive list of operation that would benefit from high throughput microinjection follows.

Genomics
DNA delivery into cells for transfection of “difficult” cell lines
DNA delivery into cells for transfection of very large DNA molecules (potentially also entire chromosomes)
Delivery into cells of known amounts of a gene construct to study the expression level of a gene of interest in different conditions (e.g., change sequences in the promoter and see how this affects gene expression in vivo)
Delivery of known amounts of DNA sequences together with known amounts of enzymes that enhance DNA recombination in order to achieve easier/more efficient stable transfection, homologues recombination and site specific mutagenesis

RNA and RNA interference (RNAi)
Delivery of known amounts of RNA for more efficient/easier RNAi (Microinjection based RNAi)
Delivery of RNA into cells for RNA silencing without the need of liposomes (treating cells with liposomes changes their membrane composition, alters the activity of calcium dependent signaling cascades and introduces a number of biases in gene expression experiments)
Efficient delivery of known amounts of RNA constructs for RNA interference into cells in order to reduce the amount of constructs used in each experiments (RNA constructs used for RNA interference are very expensive).
Delivery of known amounts of RNA molecules together with known amounts of Dicer molecules to achieve standardized, efficient, RNAi across multiple cell lines and in different conditions
Delivery of known amounts of mRNA into cells to study some aspects of gene expression regulations at the posttranscriptional level (at present these kind of studies are either impossible or extremely difficult)
Delivery of known amounts of labeled RNA to study in vivo the half life of RNAs

Proteomics
Delivery of known amounts of labeled proteins to study their half life in vivo
Delivery of labeled proteins to perform in vivo studies of protein localization
Delivery of known amounts of proteins to study their effect in vivo without the need of over expressing proteins
Delivery of known amounts of tagged proteins in order to study their interactions with other proteins in vivo without the need of over expressing them.
Delivery of labeled antibodies into living cells for in vivo immunostaining and in vivo fluorescence based western blotting

Drug Discovery
Delivery across the cell membrane of known amounts of drugs. This application would be extremely useful for drug development

Therapy
Intracellular delivery of drugs to specific subset of circulating blood cells
Intracelullar delivery of nuclec acids for gene therapy or RNAi based therapy

Cells Cryopreservation
High throughput microinjection of sugars into cells to improve cryopreservation of cells, especially oocytes

Stem Cells and transgenic organism
Delivery of DNA and/or DNA+recombination enzymes into embryonic stem cells for the development of transgenic stem cell lines
Delivery of DNA and/or DNA+recombination enzymes into zygotes/blastomers for the development of transgenic organisms

The described system can be used also to sample materials from inside the cells, to record measurements from the inner environment of the cells or from the immediate proximity of the cells (e.g. patch clamp). The same technology can be used in the field of microfluidics (e.g. generation of parallel streams of different fluids into a same microchannel) independently from the presence of cells. All these applications are also covered by the present invention.

Since the chip as described is constructed with low cost materials and low cost scalable fabrication techniques, it has very low production cost. This enables the production of disposable chips that could be used only once for a single set of experiments. This characteristic of the described technology is particularly relevant in biological applications because it eliminates contamination issues and clean up problems.

What is claimed is:

1. A microfabricated device comprising microchannels with at least one inlet and one outlet, said device comprising an immobilized needle fabricated externally or a micropipette fabricated externally or a measuring probe fabricated externally not exclusively in accordance to the techniques of microfabrication and then placed into a microfabricated system of microchannels.

2. The device of claim 1 in which a fluid containing at least a suspended cell flows into the micro channels.

3. The device of claim 2 in which the fluid flow is exploited to bring at least a cell towards the microneedle/micropipette so that the tip of microneedle/micropipette can be positioned in proximity or on the surface or inside a cell in order to withdraw or deliver a fluid to the cell.

4. The device of claim 2 in which the fluid flow is exploited to bring at least a cell towards the microneedle/micropipette/measuring probe so that the microneedle/micropipette/measuring probe can be positioned in proximity or on the surface or inside a cell in order to measure a physical or chemical property.

5. The device of claim 2 in which the fluid flow is exploited to position at least a cell in contact with the microneedle /micropipette to perform patch clamp measurements or other measurements of cell membrane potential.

6. The device of claim 2 where the immobilized microneedle/micropipette is a pulled glassed capillary or any other needle of dimension adequate to inject a cell, or the microneedle/micropipette is multibarrel.

7. The device of claim 2 where the device is made using polymers.

8. The device of claim 2 where a valve or another unit to generate backflow is placed to prevent/overcome clogging of the device.

9. A microfabricated device comprising microchannels with at least one inlet and one outlet, said device comprising an immobilized needle or micropipette or a measuring probe and at least one microfabricated valve for fluid control at the level of the microchannels.

10. The device of claim 9 in which a fluid containing at least a suspended cell flows into the micro channels.

11. The device of claim 10 in which the fluid flow is exploited to bring at least a cell towards the microneedle/micropipette so that the tip of the microneedle/micropipette can be positioned in proximity or on the surface or inside a cell in order to withdraw or deliver a fluid to the cell.

12. The device of claim 10 in which the fluid flow is exploited to bring at least a cell towards the microneedle/micropipette/measuring probe so that the microneedle/micropipette/measuring probe can be positioned in proximity or on the surface or inside a cell in order to use it as a tool to measure a physical or chemical quantity.

13. The device of claim 10 in which the fluid flow is exploited to position at least a cell in contact with the microneedle/micropipette to perform patch clamp measurements or other measurements of cell membrane potential.

14. The device of claim 10 where the immobilized microneedle/micropipette is a pulled glassed capillary or any other needle of dimension adequate to inject a cell, or the microneedle/micropipette is multibarrel.

15. The device of claim 10 where the device is made using polymers.

16. The device of claim 10 where a valve or another unit to generate backflow is placed to prevent/overcome clogging of the device.

17. A method for achieving high throughput single cells positioning with micrometric or submicrometric precision in order to establish contact or proximity between cells and a microneedle or micropipette or a measuring probe and to deliver/withdraw fluid from a cell or from the surrounding of a cell or to measure a physical/chemical quantity related to the cell, said method comprising a fluid with at least a suspended cell flowing into a microchannel and said microchannel containing at least an immobilized microneedle or micropipette or measuring probe.
